(19) United States
(12) Patent Application Publication
(10) Pub. No.: US 2008/0132726 A1
(43) Pub. Date: Jun. 5, 2008

(54) PROCESS FOR THE PRODUCTION OF QUINONE METHIDE

(76) Inventor: Vinod Kumar Rai, Bangalore (IN)

Correspondence Address: GENERAL ELECTRIC CO.;GLOBAL PATENT OPERATION, 187 Danbury Road, Suite 204, Wilton, CT, 06897-4122, US

(21) Appl. No.: 11/565,281

(22) Filed: Nov. 30, 2006

(51) Int. Cl. C07C 45/00

(52) U.S. Cl. 568/309

(57) ABSTRACT

This invention relates to a process for the production of quinone methods and more particularly to a method for process which optimizes the production of quinone methide, in particular 4-Benzylidene-2,6-di-tert-butyl-cyclohexa-2,5-dienone, while limiting the production of by products or waste.

### FIELD OF THE INVENTION

This invention relates to a process for the production of quinone methods and more particularly to a method for process which optimizes the production of quinone methide, in particular 4-Benzylidene-2,6-di-tert-butyl-cyclohexa-2,5-dienone, while limiting the production of by products or waste.

### DESCRIPTION OF THE PRIOR ART

It is known to produce quinone methide by reacting 2,6-Di-tert-butyl-phenol and benzaldehyde with dimethyl amine to get 2,6-Di-tert-butyl-4- (dimehtylamino-phenyl-methyl)-phenol to produce quinone methide (ie. 4-benzylidene-2,6-di-tert-butyl-cyclohexa-2,5-dienone). However, when this process is followed, in addition to the quinone methide, other compounds are produced which are by products, in particular, dimethyl amine and acetic anhydride are the reagents that end up as by products, ie. dimethyl acetamide and acetic acid of this process, the production of these by products contributes to both the raw material cost and to the treatment cost. Quinone methide is used as a component for retarders for styrene. The retarders prevent the undesired polymerization of styrene leading to loss of styrene monomer and loss of production owing to the deposition of polymers in or on the equipment used in the purification process. The methods involving the use of quinone methide are preferred and are an effective replacement for toxic dinitrophenols, which are heavily regulated by environmental organizations.

U.S. Pat. No. 3,660,505 teaches the reduction of quinone methides to the corresponding phenols utilizing an alkyl or aralkyl Gringnard reagent and the subsequent oxidation of the phenol with basic ferricyanide to produce a new quinone methide. followed by reactin ghe compound with a tertiary amine or a trialkylphosphine to produce an alkenyl phenol.

U.S. Pat. No. 4,032,547 discloses and claims a novel and efficient process for preparing quinone alkides form the corresponding tri-alkyl hindered phenols, utilizing ferricyanide as the secondary oxidant in combination with persulfate as the primary oxidante.

There remains a need for an alternate process for the production of quinone methide which lessens or eliminates the production of costly byproducts and waste stream generation.

### SUMMARY OF THE INVENTION

A process for the production of quinone methide, 4-Benzylidene-2,6-di-tert-butyl-cyclohexa-2,5-dienone, is disclosed, which is a “green” or environmentally positive process. The process provides for the reduction and potential elimination of waste streams and costly by products.

In one embodiment of the invention, a recyclable secondary amine catalyst, such as N,N-Di n-butyl amine or N,N-Di ni-propyl amine is used as a reactant with benzaldehye and 2,6-Di-tert-butyl phenol to produce the desired product. The process comprises the thermal cracking of the amine intermediate (2,6-Di-tert-butyl-4-(di-n-propyl amino-phenyl-methyl)-phenol or 2,6-Di-tert-butyl-4-(di-n-butyl amino-phenyl-methyl)-phenol) to result in the desired quinone methide. This process avoids the use of acetic anhydride and the amine catalyst used in the process can be recovered and reused, thereby reducing or potentially eliminating costly byproducts and waste streams.

### DETAILED DESCRIPTION OF THE INVENTION

The singular forms “a,” “an” and “the” includes plural referents unless the context clearly dictates otherwise. The endpoints of all ranges reciting the same characteristic are independently combinable and inclusive of the recited endpoint. All references are incorporated herein by reference.

The modifier “about” used in connection with a quantity is inclusive of the stated value and has the meaning dictated by the context (e.g., includes the degree of error associated with measurement of the particular quantity).

“Optional” or “optionally” means that the subsequently described event or circumstance may or may not occur, or that the subsequently identified material may or may not be present, and that the description includes instances where the event or circumstance occurs or where the material is present, and instances where the event or circumstance does not occur or the material is not present.

The present inventing relates to 4- benzylidene-2,6-di-tert-butyl-cyclohexa-2,5-dienone and a process for its production. A particular embodiment of this invention relates to the use of a recyclable secondary amine catalyst as a component of the process, thereby resulting in the elimination of the generation of a waste stream. This is due to the fact that the amine (N,N-di-n-propyl amine or N,N-di-butyl amine) produced in accordance with the present invention is recovered by distillation along with small amounts of benzaldehyde and 2,6-tert-butyl phenol and can be recycled and used again the process. This present process provides for the elimination the production of acetic acid and dimethyl acetamide, which are by products of currently used processes. By eliminating these by products and thereby the waste steam generation, the present process provides and economic as well as environmental advantage, as there is a reduction in a the raw material costs as well as the treatment costs.

The concept in the present process is the thermal cracking of the amine intermediate (2,6-Di-tert-butyl-4(di-n-propyl amino-phenyl-methyl)-phenol) to get the desired quinone methide and the starting secondary amine. In the present process, a secondary amine catalyst is reacted with benzaldehye to form the hemi aminal. Thereafter, nucleophilic substitution of 2,6-Di-tert-butyl-phenol proceeds, and results in the elimination of the amine catalyst.

The secondary amine catalyst can be chosen from any of the n-alkyl secondary amines, including any with the general formula R₁-NH-R₂ wherein R₁ and R₂ are any alkyl, including, but not limited to, methyl, ethyl, propyl, butyl, in addition to N,N-Di-n-butyl amine, N,N-Di-ethyl amine, N,N-Di-methyl-butyl amine, and N,N-Di-n-propyl amine, with the preferred secondary amines comprising N,N-Di-n butyl amine and N,N-DI-n-propyl amine. These two secondary amines give provide better conversions compared to lower n-alkyl secondary amines. Although both of these perform equally well with respect to conversion within the present process, N,N-Di-n-propyl amine is the preferred embodiment due to the ease of the reaction. Upon completion of the process, the amine catalyst, such as Di-n-propyl amine, can be recovered and recycled for use again.

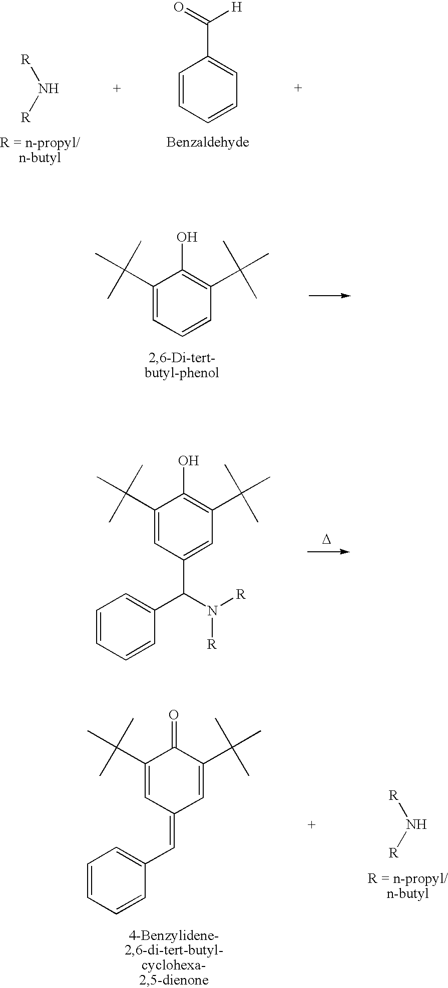

The basic process call for the reaction of the secondary amine, benzaldehyde and 2,6-Di-tert-butyl phenol to be reacted together. In a preferred embodiment, the reaction takes place in an oil bath. The reaction takes place over a period of time, from about 1 to about 30 hours, preferably from about 4 to about 14 hours. The reaction can be carried out at temperatures of from about 60° C. to about 150° C., preferably from about 90° C. to about 120° C.

Over the course of the reaction, the pressure may be applied, while the temperature remains relatively constant. The reaction may proceed by reacting and potentially stirring the initial components for period of time, from about 1 to about 15 hours, under a set temperature of from about 60° C. to about 150° C. A vacuum of about 760 mm to about 10 mm Hg is applied, and the reaction remains at this pressure for from about 0.5 to about 3 hours. Thereafter, the vacuum is slowly lowered to from about 75 to a about 10 mm Hg, for an additional time of from about 0.5 to about 4 hours, keeping the components at a constant temperature throughout.

An alternate, and preferred embodiment is to react the initial components under a temperature of from about 90° C. to about 150° C., and initially subjecting the mixture to a vacuum of from about 250 mm Hg to about 760 mm Hg, for a period of from about 5 to about 10 hours. Thereafter, the vacuum is slowly lowered to from about 75 to about 10 mm Hg, where it remains for about 1 to about 9 hours.

An additional step may added to the previous embodiment so that there are tree stages for the reaction. For this stage, the vacuum is lowered once again, to from about 35 to about 5 mm Hg, and remains there for a period of from about 10 minutes to about 4 hours.

Upon completion of these steps the product (4-Benzylidene-2,6-di-tert-butyl-cyclohexa-2,5-dienone) content is from about 60 to about 90% in the residue with some minor impurities and starting materials, preferably from about 80 to about 90% conversion. The secondary amine obtained by thermal cracking of the amine intermediate (2,6-Di-tert-butyl-4-(di-n-propyl amino-phenyl-methyl)-phenol or 2,6-Di-tert-butyl-4-(di-n-butyl amino phenyl-methyl)-phenol) is the same secondary amine initially used in the reaction, which can be recycled and reused as an initial component of the process.

### EXAMPLE I

In a 500 ml RB flask equipped with a Dean stark attached to a water cooled relux condenser, 51.3 g of 2,6-Di-tert-Butyl phenol, 26.5 g of benzaldehyde and 46 g of N,N-Di n-propyl amine were charged. The mixture was heated under nitrogen atmosphere in an oil bath over a magnetic stirrer at 130° C. for 9.5 hrs, and subjected to stirring. Thereafter, the reaction mixture was subjected to distillation at 114° C. for 4 hrs under vacuum. The initial vacuum was 250 mm, which was for a period of 2 to 3 hours. Thereafter, the vacuum was slowly lowered to 10 mm Hg, where it remained for the completion of the reaction. The weight of the final product was 73.2 g and the weight of the distillate after the separation of water was 44 g. Quinone methide content in the product was 84.79%.

### EXAMPLE II

26.5 g benzaldehyde and 32.3 g N,N-Di-n-butyl amine were charged into a 500 ml RB flask. The mixture was held at 70° C. for 30 minutes. Thereafter, 51.5 g 2,6-Di-tert-butyl-phenol was added, and the mixture was held at 115 to 118° C. for 30 hours. The reaction mixture was subjected to distillation for the recovery of amine under 30 mm Hg. vacuum at 130° C. for 5 hours and 69.78% quinone methide was found in the product after the removal of N,N-Di-n-butyl amine by distillation.

### EXAMPLE III

21.4 g benzaldehyde and 41.2 g N,N-Di-n-butyl amine were charged into a 500 ml RB flask along with 26 g 2,6-Di-tert-butyl-phenol. The mixture was held at 100 for 12 hours. Thereafter the reaction mixture was subjected to amine distillation under 30 mm Hg vacuum at 110° C. for 1.5 hours. The vacuum was slowly lowered to 6 mm Hg, and the distillation was continued for an additional 2.0 hours. The final product in the reaction flask was showing 63.82% of quinone methide after the distillation of N,N-Di-n-butyl amine.

### EXAMPLE IV

21.5 g benzaldehyde and 32.0 g N,N-di-ethyl amine were charged into a 500 ml RB flask along with 41.5 g 2,6Di-tert-butyl-phenol. The mixture was heated in an oil bath at 130° C. and held at 130° C. for 9.5 hours. Thereafter the reaction mixture was distilled of N,N-di-ethyl amine.

While typical embodiments have been set forth for the purpose of illustration, the foregoing descriptions should not be deemed to be a limitation on the scope herein. It is apparent that numerous other forms and modifications of this invention will occur to one skilled in the art without departing form the spirit and scope herein. The appended claims and these embodiments should be construed to cover all such obvious forms and modifications that are within the true spirit and scope of the present invention.

1. A process for the production of 4-benzylidene-2,6-di-teri-butyl-cyclohexa-2,5-dienone, comprising reacting benzaldehyde, and 2,6-Di-tert-butyl-phenol in the presence of a secondary amine catalyst, which is earned out at temperatures of from about 60° C. to about 150° C.

2-3. (canceled)

4. The process of claim 1 wherein the n-alkyl secondary amine is chosen from the group consisting of N,N-Di-n-butyl amine, N,N-Di-ethyl amine, N,N-Di-methyl-butyl amine, and N,N-Di-n-propyl amine.

5. The process of claim 1 wherein the n-alkyl secondary amine is chosen from N,N-Di-methyl-butyl amine, or N,N-Di-n-propyl amine.

6. The process of claim 1 wherein an initial reaction takes place in an oil bath.

7. The process of claim 1 wherein the reaction mixture is further subjected to amine distillation.

8. The process of claim 7 wherein the distillation is carried out under vacuum and at a temperature of about 110 to about 114° C.

9. The process of claim 8 wherein the vacuum is from about 300 to about 760 mm Hg.

10. The process of claim 1 wherein the process takes place over a period of from about 1 to about 30 hours.

11. A process for the production of 4-benzylidene-2,6-di-tert-butyl-cyclohexa-2,5-dienone, comprising;
(a) reacting benzaldehyde, and 2,6-Di-tert-butyl-phenol in the presence of a secondary amine catalyst;
(b) subjecting the reaction mixture to vacuum during distillation which is conducted at a temperature of about 110 to about 114° C.; and
(c) recovering the amine catalyst and recycling it for reuse.

12. The process of claim 11 wherein process takes place over a period of from about 1 to about 30 hours, and under a vacuum of from about 300 to about 760 mm Hg.

13. The process of claim 11 wherein the product after the distillation of amine comprises from about 60 to about 90% by weight of quinone methide.

14. A process for the production of 4-benzylidene-2,6-di-tert-butyl-cyclohexa-2,5-dienone, comprising;
(a) reacting benzaldehyde, and 2,6-Di-iert-butyl-phenol in the presence of a secondary amine catalyst;
(b) subjecting the reaction mixture to vacuum of from about 250 to about 760 mm Hg for a period of from about 5 to about 10 hours;
(c) slowly lowering the vacuum to from about 75 to about 10 mm Hg, for a period of from about 1 to about 9 hours; and
(d) recovering the distilled secondary amine and recycling it for reuse.

15. The process of claim 14 wherein the product after the distillation of amine comprises about 60 to about 90% by weight quinone methide.

16. The process of claim 14 wherein the product after the distillation of amine comprises from about 80 to about 90% by weight quinone methide.

17. A process for the production of 4-benzylidene-2,6-di-tert-butyl-cyclohexa-2,5-dienone, comprising;
(a) reacting benzaldehyde, and 2,6-Di-tert-butyl-phenol in the presence of a secondary amine catalyst;
(b) subjecting the reaction to vacuum of from about 250 to about 760 mm Hg for a period of from about 5 to about 10 hours;
(c) slowly lowering the vacuum to from about 75 to about 10 mm Hg, for a period of from about 1 to about 9 hours;
(d) slowly lowering the vacuum to from about 35 to about 5 mm Hg for a period of from about 10 minutes to about 4 hours; and
(e) recovering the secondary amine and recycling it for reuse,

18. The process of claim 17 wherein the product after the distillation of amine comprises about 60 to about 90% by weight quinone methide.

19. The process of claim 17 wherein the product after the distillation of amine comprises from about 80 to about 90% by weight quinone methide.
